(19) United States
(12) Patent Application Publication
(10) Pub. No.: US 2013/0165785 A1
(43) Pub. Date: Jun. 27, 2013

(54) METHOD AND APPARATUS FOR APERTURE SELECTION IN ULTRASOUND IMAGING

(71) Applicant: Brian Anthony Lause, Milwaukee, WI (US)

(72) Inventor: Brian Anthony Lause, Milwaukee, WI (US)

(73) Assignee: General Electric Company, Schenectady, NY (US)

(21) Appl. No.: 13/333,586

(22) Filed: Dec. 21, 2011

(51) Int. Cl. A61B 8/12

(52) U.S. Cl. 600/443

(57) ABSTRACT

A method for controlling an ultrasound imaging system includes defining a sample volume gate on a two-dimensional (2D) ultrasound image, the sample volume gate defining a location at which flow is to be estimated, automatically calculating a SNR for an initial transmit and receive steering position (aperture location) and aperture size, automatically calculating a SNR for a different second transmit and receive steering position (aperture location) and aperture size, automatically comparing the SNR for the first set of apertures to the SNR for the second set of apertures, and automatically adjusting the steering angle and an aperture size of an ultrasound probe's transmit and receive events based on the comparison.

### BACKGROUND OF THE INVENTION

The subject matter disclosed herein relates generally to ultrasound imaging systems, and more particularly, to a method and apparatus for improving an aperture selection of an ultrasound probe.

Ultrasound imaging systems may be utilized to measure the velocity of blood flow using spectral Doppler techniques. In operation, an ultrasound probe may transmit pulsed wave (PW) or continuous wave (CW) Doppler waveforms into an object and receive backscattered and reflected ultrasonic echoes. To measure the blood flow characteristics, returning ultrasound waves are compared to a frequency reference to determine the frequency shift imparted to the returning waves by flowing scatterers, such as for example, blood cells. The frequency shift translates into the velocity of the blood flow.

PW or CW Doppler waveforms may be computed and displayed in real-time as a spectrum or spectral image of the Doppler frequency (or velocity) versus time with the gray-scale intensity (or color) modulated by the spectral power. Each spectral line represents an instantaneous measurement of blood flow within a sampling gate. To identify a specific location to acquire the spectral Doppler information, a user typically places an indicator, also referred to as a sampling gate, on the B-mode image that indicates a position at which the user desires to acquire the blood flow velocity. A user may then manually steer the ultrasound transmit beam to any desired transmit angle.

In medical ultrasound imaging it is desirable to optimize the signal-to-noise ratio (SNR). SNR is the ratio of the amplitude of the sound waves to the undesired system and acoustic noise. In spectral Doppler imaging, the SNR is related to the angle at which the ultrasound waves intercept the blood cells. In operation, an optimal Doppler angle is achieved when the sound waves are parallel to the flow of blood in the blood vessel. As the angle of the sound waves intercepting the blood flow increases from the optimal parallel angle, the received signal decreases. For example, if the ultrasound probe is positioned approximately perpendicular to the flow of blood cells, the sound waves intercept the blood cells at approximately 90 degrees resulting in no Doppler frequency shift and therefore no useful signal. Optionally, if the ultrasound probe is positioned approximately parallel to the flow of blood cells, the Doppler shift and therefore returning signal is relatively high. Thus, for maximum signal it is desirable to position the ultrasound probe such that the sound waves emit in a direction that is parallel to the flow of blood cells.

However, many blood vessels are not perpendicular to a surface of the patient's skin. Therefore, in many cases, the ultrasound probe can not be positioned in such a manner that the sound waves emit in a direction that is parallel to the flow of blood in the blood vessel to achieve the maximum signal. To improve the SNR, at least some known ultrasound imaging systems enable the operator to manually change the steering angle of the transmitted beam. However, increasing the steering angle of the sound waves decreases the acoustic efficiency of the imaging system. More specifically, when the sound waves are emitted in a substantially perpendicular direction from the element face, the acoustic elements in the ultrasound probe have the highest efficiency in translating electrical energy to the acoustic energy and translating acoustic energy back to electrical energy. However, as the steering angle of the sound waves is moved in a direction to a greater angle, the efficiency of the imaging system decreases.

Accordingly, in operation the user balances two competing interests: positioning the ultrasound probe to be as parallel to the blood flow as possible; and positioning the ultrasound probe to emit sound waves in a substantially perpendicular direction to the face of the ultrasound probe in order to maximize SNR. As a result, it may be difficult or time consuming for an operator to position the ultrasound probe, and manually adjust the steering angle of the sound waves emitted from the ultrasound probe, in such a manner that both competing interests are taken into account and the optimal SNR is arrived at.

### BRIEF DESCRIPTION OF THE INVENTION

In one embodiment, a method for controlling an ultrasound imaging system is provided. The method includes defining a sample volume gate on a two-dimensional (2D) ultrasound image, the sample volume gate defining a location at which flow is to be estimated, automatically calculating a SNR with an initial aperture size and position, automatically calculating a SNR with a different aperture size and or position, automatically comparing the SNR with the first aperture to the SNR with the second steering aperture, and automatically adjusting the steering angle (aperture location) and aperture size for the transmit and receive beams of an ultrasound probe based on the comparison.

In another embodiment, an ultrasound imaging system is provided. The ultrasound imaging system includes an ultrasound probe having a transducer emitting ultrasound beams into a patient, the ultrasound probe acquiring a volume of ultrasound data including a blood vessel, a user interface for defining a sample volume within the blood vessel, and a processor. The processor is configured to automatically calculate a SNR with an initial aperture size and location, automatically calculate a SNR with a different second aperture size and location, automatically compare the SNR with the first aperture to the SNR with the second aperture, and automatically adjust the steering angle (aperture location) and an aperture size for the transmit and receive beams of the ultrasound probe based on the comparison.

In a further embodiment, a non-transitory computer readable medium is provided. The non-transitory computer readable medium is programmed to instruct a computer to automatically calculate a SNR for an initial aperture size and location, automatically calculate a SNR for a second aperture size and location, automatically compare the SNR at the first steering position to the SNR at the second steering position, and automatically adjust the steering angle (aperture location) and an aperture size of the ultrasound probe based on the comparison.

### BRIEF DESCRIPTION OF THE DRAWINGS

FIG. 1 is a block diagram of a medical imaging system formed in accordance with various embodiments.

FIG. 2 is flowchart of an exemplary method of improving the signal-to-noise (SNR) of acquired spectral data on an ultrasound imaging system.

FIG. 3 is diagram of an ultrasound scan that may be performed in accordance with various embodiments.

FIG. 4 is a B-mode image that may be generated in accordance with various embodiments.

FIG. 5 is a spectrogram that may be generated in accordance with various embodiments.

FIG. 6 is flowchart of a portion of the method shown in FIG. 1 in accordance with various embodiments.

FIG. 7 is a diagram of an ultrasound scan that may be performed in accordance with various embodiments.

FIG. 8 is another spectrogram that may be generated in accordance with various embodiments.

FIG. 9 is a block diagram illustrating a portion of the imaging system shown in FIG. 1 in accordance with various embodiments.

### DETAILED DESCRIPTION OF THE INVENTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrase “reconstructing an image” is not intended to exclude embodiments of the present invention in which data representing an image is generated, but a viewable image is not. Therefore, as used herein the term “image” broadly refers to both viewable images and data representing a viewable image. However, many embodiments generate, or are configured to generate, at least one viewable image.

Described herein are various embodiments for automatically adjusting a size and a location of an ultrasound probe active aperture to improve the signal-to-noise (SNR) for a particular patient, exam, and probe location, being examined. In operation, the system of various embodiments automatically identifies a plurality of aperture sizes and locations and automatically selects the aperture size and location for both the transmit and receive apertures that provides the highest SNR capabilities. At least one technical effect is to automatically identify a beam steering angle that results in the optimal SNR.

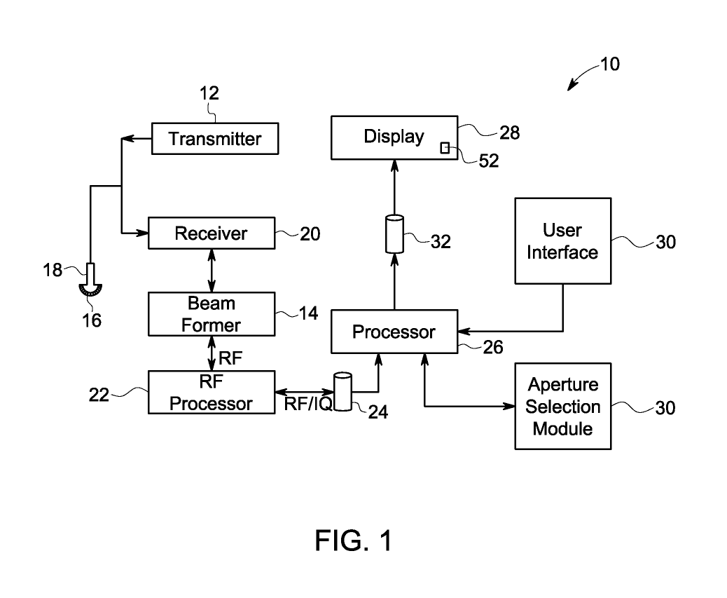

Various embodiments described herein may be implemented in an ultrasound system as shown in FIG. 1. More specifically, FIG. 1 is a block diagram of an exemplary ultrasound imaging system 10 that is constructed in accordance with various embodiments. The ultrasound system 10 is capable of electrical or mechanical steering of a soundbeam (such as in 3D space) and is configurable to acquire information (e.g., image slices) corresponding to a plurality of 2D representations or images of a sample volume location (SVL) in a subject or patient, which may be defined or adjusted as described in more detail herein. The ultrasound system 10 is configurable to acquire 2D images in one or more planes of orientation. The ultrasound system 10 may be embodied in a small-sized system, such as laptop computer, a portable imaging system, a pocket sized system as well as in a larger console-type system.

The ultrasound system 10 includes a transmitter 12 that, under the guidance of a beamformer 14, drives an array of elements 16 (e.g., piezoelectric elements) within a probe 18 to emit pulsed or continuous ultrasonic signals, i.e. sound waves, into a body. A variety of geometries may be used. The sound waves are back-scattered from structures in the body, like blood cells flowing through a blood vessel, to produce echoes that return to the elements 16. The echoes are received by a receiver 20. The received echoes are processed by the beamformer 14, which performs receive beamforming and outputs an RF signal. The RF signal then passes through an RF processor 22. Optionally, the RF processor 22 may include a complex demodulator (not shown) that demodulates the RF signal to form IQ data pairs representative of the echo signals. The RF or IQ signal data may then be routed directly to a buffer 24 for storage.

In the above-described embodiment, the beamformer 14 operates as a transmit and receive beamformer. Optionally, the probe 18 includes a 2D array with sub-aperture receive beamforming inside the probe 18. The beamformer 14 may delay, apodize and/or sum each electrical signal with other electrical signals received from the probe 18. The summed signals represent spatially focused receive echoes. The summed signals are output from the beamformer 14 to the RF processor 22. The RF processor 22 may generate different data types, e.g. B-mode, color Doppler (velocity/power/variance), tissue Doppler (velocity), and Doppler energy, for multiple scan planes or different scanning patterns. For example, the RF processor 22 may generate blood flow Doppler data for multi-scan planes. The RF processor 22 gathers the information (e.g. I/Q, B-mode, color Doppler, tissue Doppler, and Doppler energy information) related to multiple data slices/receive events and stores the data information, which may include time stamp and orientation/rotation information, in the buffer 24.

The ultrasound system 10 also includes a processor 26 to process the acquired ultrasound information (e.g., RF signal data or IQ data pairs) and prepare frames and or volumes of ultrasound information for display on a display 28. The processor 26 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in the buffer 24 during a scanning session and then processed and displayed in an off-line operation.

The processor 26 is connected to a user interface 30 that may control operation of the processor 26 as explained below in more detail. The display 28 may include one or more monitors that present patient information, including diagnostic ultrasound images to the user for diagnosis and analysis. The buffer 24 and/or a memory 32 may store two-dimensional (2D) or three-dimensional (3D) data sets of the ultrasound data, where such 2D and 3D data sets are accessed to present 2D (and/or 3D images). The images may be modified and the display settings of the display 28 may also be manually adjusted using the user interface 30.

In various embodiments, the ultrasound system 10 also includes an automatic aperture selection module 50. The automatic aperture selection module 50 may be programmed to identify a size and a location of an aperture based on inputs received from the probe 18. The aperture selection module 50 may be software running on the processor 26 or hardware provided as part of the processor 26. More specifically, the aperture selection module 50 may be embodied as a set of instructions or program that is executed by the processor 26. The program instructions may be written in any suitable computer language, e.g., Matlab. The processor 26 may therefore be any one or a combination of suitably appropriate processing systems, such as, for example, a microprocessor, a digital signal processor, and a field programmable logic array, among others. The processing system may be embodied as any suitable computing device, e.g., a computer, personal digital assistant (PDA), laptop computer, notebook computer, a hard-drive based device, or any device that can receive, send, and store data.

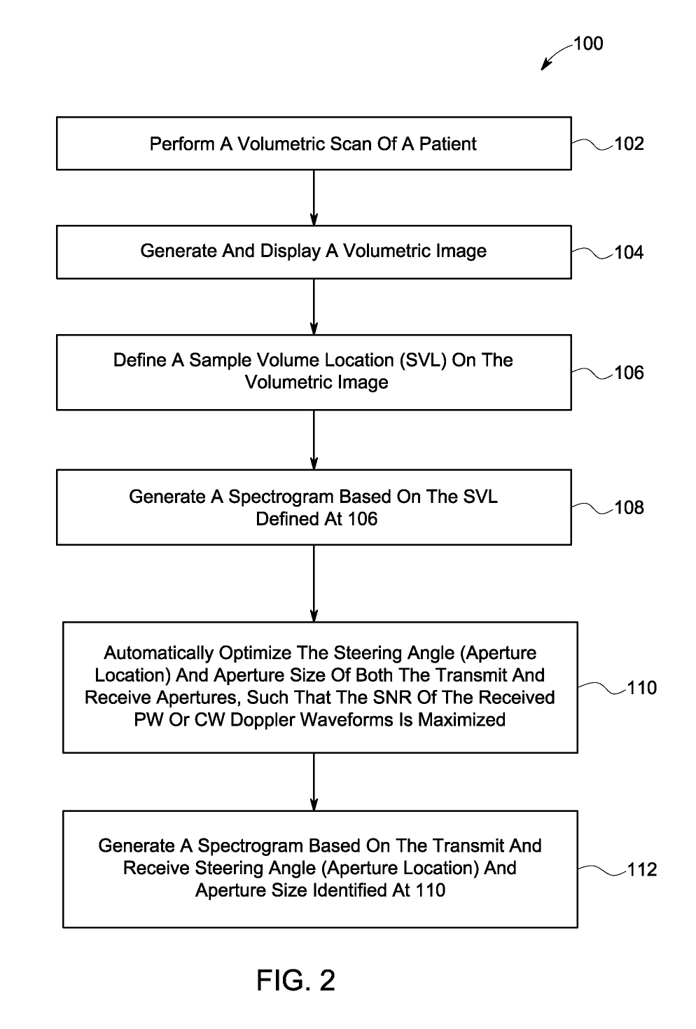

FIG. 2 is a flowchart of an exemplary method 100 that may be performed by the imaging system 10 shown in FIG. 1. In various embodiments, the method 100 may be implemented using the aperture selection module 50 also shown in FIG. 1. More specifically, the method 100 may be provided as a non-transitory computer-readable medium or media having instructions recorded thereon for directing the processor 26 to perform one or more embodiments of the methods described herein. The medium or media may be any type of CD-ROM, DVD, floppy disk, hard disk, optical disk, flash RAM drive, or other type of computer-readable medium or a combination thereof.

In operation, in various embodiments, the method 100 enables the ultrasound system 10 to automatically generate either a stronger Doppler signal or a Doppler signal with less noise more quickly than could be manually obtained by the user. Moreover, the method 100 may search for the strong signal using more options than the user currently has available.

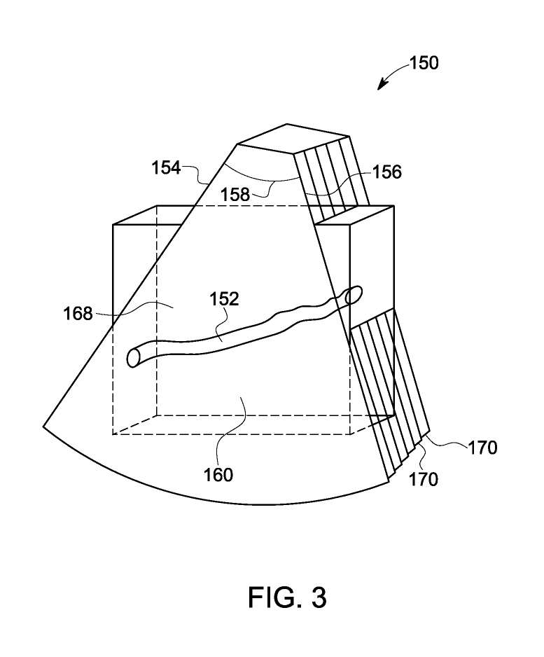

At 102, a volumetric scan of a patient is performed. The volumetric scan may be performed by operating the probe 18 to emit, B-mode waveforms and generate image (not spectral) data. In operation, the user may position the probe 18 on an area of the patient to be imaged. For example, as shown in FIG. 3, the probe 18 may be used to acquire a volume 150 having blood vessel 152 therein. The volume 150 is defined by a plurality of sector shaped cross-sections with radial borders 154 and 156 diverging from one another at an angle 158. The probe 18 (shown in FIG. 1) electronically focuses and directs ultrasound firings longitudinally/in-plane/laterally to scan along adjacent scan lines in each scan plane and electronically or mechanically focuses and directs ultrasound firings elevationally to scan adjacent scan planes. The scan planes obtained by the probe 18 are stored in the memory 24 or 32 and are scan converted from spherical or cylindrical coordinates to Cartesian coordinates by the volume scan converter processor 26. A volume including the scan planes is output from the processor as a rendering region 160. The rendering region 160 is formed from multiple adjacent scan planes.

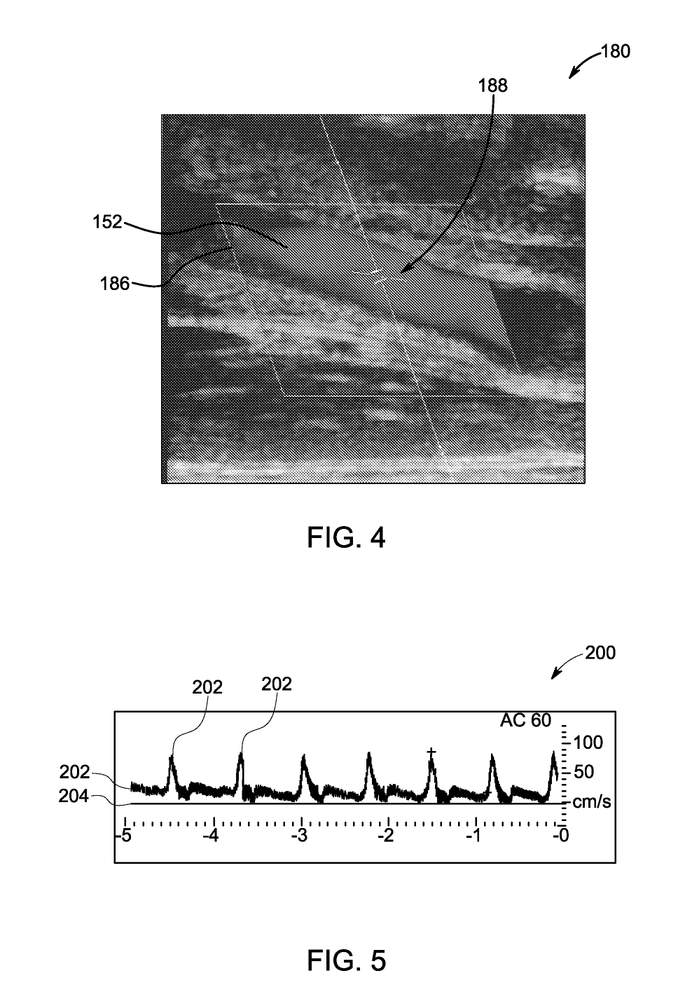

Referring again to FIG. 2, at 104, an image based on the volumetric scan is generated and displayed, such as on the display 28 shown in FIG. 1. In various embodiments, the image may be a B-mode image with or without a color Doppler image, or any other type of image that shows the vessel 152. Moreover, the image may be a two-dimensional (2D) image or a three-dimensional (3D) image. For example, FIG. 4 illustrates an exemplary 2D B-mode image 180 that may be generated based on information acquired from the 2D probe 18 and may be displayed on the display 28. The user may adjust the area to be imaged by manually viewing a cursor (not shown) that is displayed on the display 28. The user may then manually reposition the probe 18 to the desired area while concurrently viewing the location of the cursor on the B-mode image 180. Thus, the operator may ascertain the location of the probe 18 while viewing the location of the cursor on the display 28.

Referring again to FIG. 2, at 106 a user defines the sample volume location (SVL) on the image generated at 104. For example, referring again to FIG. 4, the user may define a SVL 186 using, for example, the user interface 30. A sample volume gate 188 may be placed and moved within the SVL 186 to a desired location where the user wishes to evaluate flow. FIG. 4 also illustrates an exemplary steered beam 189 that denotes the current steering angle of the ultrasound beams. In various embodiments, the SVL 186 and/or the sample volume gate 188 may be placed at the edges of the B-mode image 180. In various embodiments, the user may also change a size and a shape of the sample volume gate 188 within the displayed B-mode image 180. The sample volume gate 188 is illustrated on the B-mode image 180, using a pair of lines, however it should be realized that the sample volume gate 188 may be a single point. More specifically, the user may select a single point in the blood vessel 152 to acquire flow information. Moreover, in various embodiments, the user does not need to define the SVL 186. Rather, as described above, the user may simply point to a single area in the blood vessel 152 using the user interface 30 to define the sample volume gate 188.

Referring again to FIG. 2, at 108 a spectrogram is generated based on the sample volume gate 188 defined at 106. More specifically, the processed PW or CW Doppler received echoes are computed and displayed in real-time as a spectrogram or spectral image of Doppler frequency (or velocity) versus time with the gray-scale intensity (or color) modulated by the spectral power. For example, FIG. 5 illustrates an exemplary spectrogram 200 that may be generated at 108. As shown in FIG. 5, the spectrogram 200 is generated using a plurality of spectral lines 202 wherein each spectral line 202 represents an instantaneous measurement of blood flow within the sampling gate 188. All of the spectral lines 202 taken together therefore form the spectrogram or spectrum 200. Accordingly, each vertical line or spectral line 202 in the spectrogram 200 corresponds to a Doppler frequency spectrum at a given time instant. Positive Doppler frequencies correspond to flow towards the probe 18, and negative frequencies correspond to flow away from the probe 18, as referenced by a baseline 204 at frequency equal to zero.

Referring again to FIG. 2, at 110, the steering angle of transmit and or receive beam (aperture location) as well as the aperture size is adjusted, or automatically optimized, such that the SNR of PW or CW Doppler waveforms received at the probe 18 is increased or maximized. In various embodiments, optimization of the steering angle may be manually initiated by the operator. For example, the display 28 may include an icon 52 (shown in FIG. 1). In operation, the user may operate the user interface 30 to activate the optimization of the steering angle by selecting the icon 52. Once the user has selected the icon 52, the aperture selection module 50 automatically selects an aperture size and an aperture location that increases or maximizes the SNR of the PW or CW Doppler received waveforms. More specifically, in one embodiment, the aperture selection module 50 is programmed to automatically select an optimal steering angle that increases or maximizes the SNR.

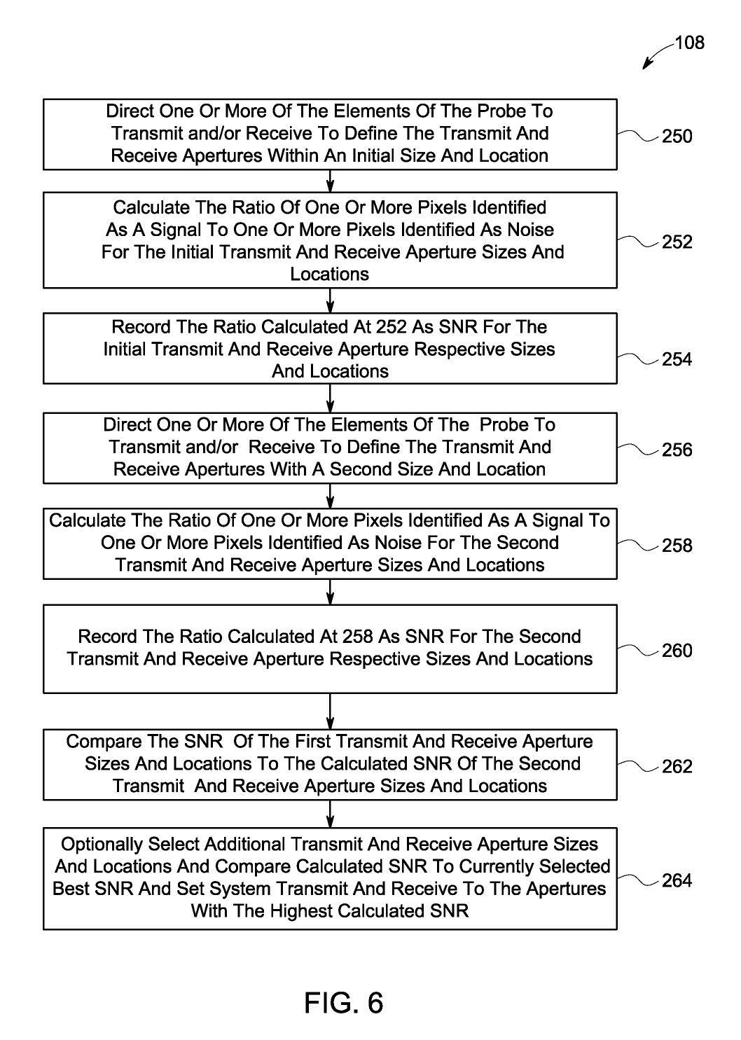

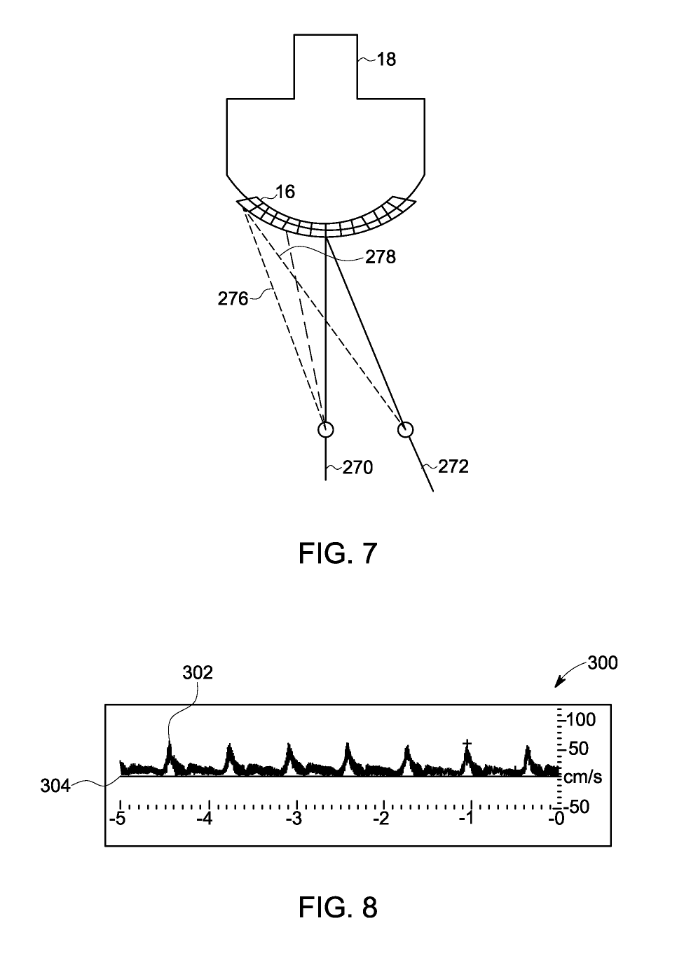

FIG. 6 is a flowchart illustrating the method step 110 shown in FIG. 2. In the exemplary embodiment, at 250 the automatic aperture selection module 50 is configured to direct one or more of the elements 16 of the probe 18 to transmit and/or receive. In this way both a transmit and receive aperture contain one or more elements is defined. The transmit and receive apertures can be defined with a center position as well as size (in units of length or elements). For example, FIG. 7 is a diagram of an ultrasound scan that may be performed in accordance with various embodiments at step 110. The ultrasound beam may be, for example, a non-steered beam 270 (e.g. a beam that is perpendicular to the face of the probe 18) or a steered beam 272 (e.g. a beam that is not perpendicular to the face of the probe 18). The non-steered beam 270 may be an ultrasound beam transmitted in a direction generally along the major axis of the probe 18. The steered beam 272 may be an ultrasound beam transmitted in a direction other than that of the non-steered beam 270. For example, the steered beam 272 may have a propagation path that is 10 degrees from the propagation path of a non-steered beam 270. In various embodiments, the methods described herein, when selecting apertures, generates a beam which intersects with the defined center location of the SVG. The beam steering, defined either by the user or by the SNR optimization selects steered beams that intersect at the SVG.

At 252, ultrasound information acquired from either the non-steered beam 270 or the steered beam, if selected, is analyzed to determine one or more pixels selected as ‘signal’ in the computed spectrum. For example, referring again to FIG. 5, assume that a pixel within the line 274 represents a pixel selected as a ‘signal’ in the non-steered beam 270. A pixel within the line 274 may be identified as the ‘signal’ for example, by identifying the pixel intensity values for each of the pixels within the line 274 and then selecting the pixel having the highest pixel intensity value. The remaining pixels or a subset of the remaining pixels in the line 274 may then be classified as ‘noise’. The signal-to-noise-ratio (SNR) of the calculated spectral line, e.g. line 274 is then calculated. In various embodiments, the SNR of the spectral line 274 may be calculated by dividing the average or maximum intensity value of the pixels labeled ‘signal’ within spectral line 274 by an average or maximum of the pixel intensity values of the pixels labeled ‘noise’ in the spectral line 274.

At 254, the SNR of calculated spectral line 274 and the transmit and receive apertures and angles at which the ultrasound information was acquired is recorded.

At 256, the automatic aperture selection module 50 is configured to direct one or more of the elements 16 of the probe 18 to transmit and/or receive to define the transmit and receive apertures with a second size and location. In this way both a transmit and receive aperture contain one or more elements is defined with a second size at a second location. The transmit and receive apertures can be defined with a center position as well as size (in units of length or elements. For example, referring again to FIG. 7, the ultrasound beam may be, for example, a second ultrasound beam 276 that is 10 degrees from the propagation path of the non-steered beam 270. Optionally, a second beam 278 may be, for example, a second ultrasound beam 278 that is 10 degrees from the propagation path of the steered beam 272. Thus, in the exemplary embodiment, the scan step size between the first and second scan positions is approximately 10 degrees. However, other steering angles may be used.

At 258, ultrasound information acquired from either the non-steered beam 270 or the steered beam, if selected, is analyzed to determine one or more pixels selected as ‘signal’ in the computed spectrum. Ultrasound information acquired from either the non-steered beam 276 or the steered beam 278, if selected is analyzed to determine the brightest pixel 280. It should be realized that the spectrogram shown in FIG. 5 is continuously updating, therefore the brightest pixel 280, at the second scan position, may not be shown concurrently with the brightest pixel 274 at the first scan position. It should be further realized that in the exemplary embodiment, the methods described at step 108 are performed by the automatic aperture selection module 50 without user input, and therefore in various embodiments, spectrograms showing the brightest pixels, or any other information, may not be generated and displayed at 108. Therefore, the pixels 274 and 280 are only shown in FIG. 5 to more clearly describe the various embodiments described herein.

In operation, the brightest pixel 280, at the second scan position, may be identified for example, by identifying the pixel intensity values for each of the pixels within the sampling gate 188, at the second scan position, and then selecting the pixel having the highest pixel intensity value. The remaining pixels in the sampling gate 188, at the second scan position, may then be classified as noise. The SNR of the brightest pixel, e.g. the pixel 280, at the second scan position is then calculated. In various embodiments, the SNR of the pixel 280 may be calculated by dividing the intensity value of the pixel 280 by an average of the pixel intensity values of the remaining pixels in the sampling gate 188 at the second scan position.

At 260, the SNR of the brightest pixel, e.g. the pixel 280 and the angle at which the ultrasound information at which the pixel 280 was acquired is recorded.

At 262, the SNR of the pixel 274 is compared to the SNR of the pixel 280 to generate a third or revised scan position. For example, assume that the SNR of the pixel 280 is greater than the SNR of the pixel 274. Accordingly, the automatic aperture selection module 50 may determine that the SNR of the ultrasound beams may be increased, or improved, by steering the ultrasound beam “left” from the original or starting scan position. More specifically, the automatic aperture selection module 50 may determine that the SNR of ultrasound beams is increased when the ultrasound beam are steered at the angle as compared to the ultrasound beams 270, as described in more detail below.

At 264, the automatic aperture selection module 50 is configured to continuously scan at various angles to identify the aperture angle and size that results in ultrasound beams having the highest SNR. More specifically, for any given aperture size and location (angle), a SNR may be calculated. Moreover, the probe 18 defines a 2D space that indicates every possible aperture angle and every possible aperture location possible. Accordingly, in operation the automatic aperture selection module 50 is configured to search this space for the best SNR. For example, at 262, the automatic aperture selection module 50 determined that the SNR of the ultrasound beams, indicated by the line 276, were greater than the SNR of the ultrasound beams indicated by the line 270. Accordingly, based on a priori information, e.g. that the SNR is improved by steering the ultrasound beams away from the first scan position, denoted by the line 270 toward the second scan position denoted by the line 276, the automatic aperture selection module 50 may perform at scan at a third position that is between the first and second scan positions, e.g. 5 degrees from the first and second scan positions. The automatic aperture selection module 50 then identifies the brightest pixel, at the third scan position and compares the SNR of the brightest pixel at the third scan position to the SNR of the brightest pixels at the first and second scan positions. In this manner, the aperture selection module 50 iteratively scans the blood vessel 152 to steer the probe 18 to various scan angles, acquire the SNR information at the various scan angles, and identify which scan angle has the highest SNR.

Accordingly, in various embodiments, the aperture selection module 50 is configured to continuously scan at various angles based on a step size, for example, the aperture selection module 50 may scan the blood vessel 152 using five degrees step sizes, 10 degree step sizes, etc. Moreover, once the aperture selection module 50 has identified the steering angle that results in an increased SNR using the scan steps, the aperture selection module 50 may focus in on the area by scanning intermediate areas using smaller step sizes until the aperture selection module 50 identifies the beam steering angle that results in the highest SNR. In various embodiments, moving the beam steering angle using various angles steps reduces the overall time required to identify the steering angle at which the ultrasound beams have the highest SNR.

Optionally, the aperture selection module 50 may be configured to scan the entire 2D scan plane 254 using some predetermined step size. For example, the aperture selection module 50 may be configured to perform the 3D sweep in one degree increments. The aperture selection module 50 may then identify the brightest pixel at each one degree increment, determine the SNR for the brightest pixel at each one degree increment, and then compare the SNR for each pixel at each one degree increment to identify the beam steering angle that produces the highest SNR signals. Accordingly, in various embodiments, the scan steps of the search area may have a very fine resolution, or a coarse resolution to reduce the scan time. Thus, at 264, the aperture selection module 50 is configured to identify the steering angle and aperture size that produces ultrasound beams having the highest SNR.

In various embodiments, additional transmit beams may be interleaved temporally with the transmit beams utilized to determine the steering angle and aperture size having the highest SNR as described above. For example, because a certain position in the image is scanned, using the sample volume gate 158, the depth of the scanned position is known. Moreover, in various embodiments, the ultrasound beams may be emitted in relatively short bursts having relatively short time duration. Accordingly, there is a time duration between bursts of ultrasound beams wherein the ultrasound system is inactive, such that the ultrasound system 10 is not transmitting ultrasound beams to the target.

In operation, the distance between one transmit firing and a second transmit firing is referred to as a pulse repetition time (PRT). 1/PRT=a pulse repetition frequency (PRF). The PRF may be modified by the operator by changing the sampling frequency. For example, according to the Nyquist law, if a certain frequency is desired to be sampled or to identify a given frequency, sampling must be performed at twice the desired frequency or twice the frequency to be identified. Therefore, the PRF may be driven higher to observe higher velocities in the body. However, if the PRF is relatively low, the resultant images may have aliasing artifacts. Accordingly, in various embodiments, the operator may select to increase the PRT and thus reduce potential aliasing artifacts and increase the resolution of a generated image.

Referring again to FIG. 2, at 112, a spectrogram is generated based on the transmit and receive steering angle (aperture location) and aperture size identified at 110. More specifically, the PW or CW Doppler waveform is computed and displayed in real-time as a spectrum or spectral image of Doppler frequency (or velocity) versus time with the gray-scale intensity (or color) modulated by the spectral power using the aperture angle and size defined at 188.

For example, FIG. 8 illustrates an exemplary spectrogram 300 that may be generated at 112. As shown in FIG. 8, each spectral line 302 represents an instantaneous measurement of blood flow within the sampling gate 188. All of the spectral lines 302 taken together therefore form the spectrogram or spectrum 300. Accordingly, each vertical line or spectral line 302 in the spectrogram 300 corresponds to a Doppler frequency spectrum at a given time instant at the revised, or adjusted, scanning position determined at 264. Positive Doppler frequencies correspond to flow towards the probe 18, and negative frequencies correspond to flow away from the probe 18, as referenced by a baseline 304 at frequency equal to zero. In various embodiments, the spectrogram 300 may be continuously updating as the aperture selection module 50 is continuously scanning at various angles to identify the aperture size and location that produces higher or the highest SNR. Thus, various embodiments provide a method and system that enables an operator to automatically change the aperture size and location of the ultrasound probe to increase SNR and therefore automatically improve the image quality.

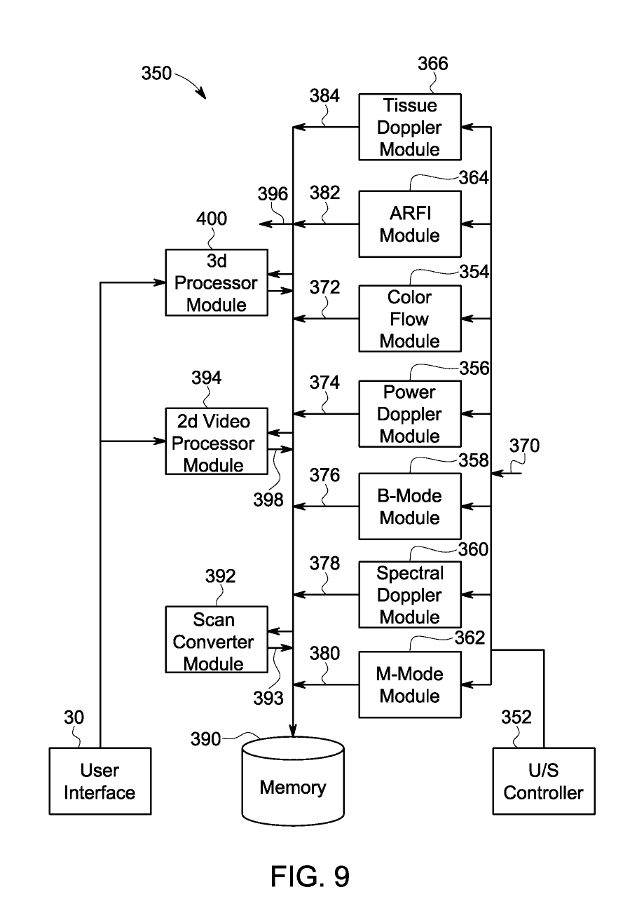

The various components of the ultrasound system 10 may have different configurations. For example, FIG. 9 illustrates an exemplary block diagram of an ultrasound processor module 350, which may be embodied as a portion of the processor 26 shown in FIG. 1. The ultrasound processor module 350 is illustrated conceptually as a collection of sub-modules, but may be implemented utilizing any combination of dedicated hardware boards, DSPs, processors, etc. Alternatively, the sub-modules of FIG. 9 may be implemented utilizing an off-the-shelf PC with a single processor or multiple processors, with the functional operations distributed between the processors. As a further option, the sub-modules of FIG. 9 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 9 may be controlled by a local ultrasound controller 352 or by the processor 26. The sub-modules 354-366 perform mid-processor operations. The ultrasound processor module 350 may receive ultrasound data 370 in one of several forms. In the embodiment of FIG. 9, the received ultrasound data 370 constitutes I,Q data pairs representing the real and imaginary components associated with each data sample. The I,Q data pairs are provided to one or more of a color-flow sub-module 354, a power Doppler sub-module 356, a B-mode sub-module 358, aspectral Doppler sub-module 360 and an M-mode sub-module 362. Optionally, other sub-modules may be included such as an Acoustic Radiation Force Impulse (ARFI) sub-module 364 and a Tissue Doppler (TDE) sub-module 366, among others.

Each of sub-modules 354-366 are configured to process the I,Q data pairs in a corresponding manner to generate color-flow data 372, power Doppler data 374, B-mode data 376, spectral Doppler data 378, M-mode data 380, ARFI data 382, and tissue Doppler data 384, all of which may be stored in a memory 390 (or memory 24 or memory 32 shown in FIG. 1) temporarily before subsequent processing. For example, the B-mode sub-module 358 may generate B-mode data 376 including a plurality of B-mode image planes, such as the image 180 shown in FIG. 4.

The data 372-484 may be stored, for example, as sets of vector data values, where each set defines an individual ultrasound image frame. The vector data values are generally organized based on the polar coordinate system.

A scan converter sub-module 392 accesses and obtains from the memory 390 the vector data values associated with an image frame and converts the set of vector data values to Cartesian coordinates to generate an ultrasound image frame 393 formatted for display. The ultrasound image frames 393 generated by the scan converter module 392 may be provided back to the memory 390 for subsequent processing or may be provided to the memory 24 or the memory 32.

Once the scan converter sub-module 392 generates the ultrasound image frames 393 associated with, for example, B-mode image data, and the like, the image frames 393 may be restored in the memory 390 or communicated over a bus 396 to a database (not shown), the memory 24, and the memory 32 and/or to other processors.

The scan converted data may be converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 28 (shown in FIG. 1), which may include one or more monitors or windows of the display, to display the image frame. The image displayed in the display 28 is produced from image frames of data in which each datum indicates the intensity or brightness of a respective pixel in the display.

Referring again to FIG. 9, a 2D video processor sub-module 394 combines one or more of the frames generated from the different types of ultrasound information. For example, the 2D video processor sub-module 394 may combine a different image frames by mapping one type of data to a grey map and mapping the other type of data to a color map for video display. In the final displayed image, color pixel data may be superimposed on the grey scale pixel data to form a single multi-mode image frame 398 (e.g., functional image) that is again re-stored in the memory 390 or communicated over the bus 396. Successive frames of images may be stored as a cine loop in the memory 390 or memory 390. The cine loop represents a first in, first out circular image buffer to capture image data that is displayed to the user. The user may freeze the cine loop by entering a freeze command at the user interface 30. The user interface 30 may include, for example, a keyboard and mouse and all other input controls associated with inputting information into the ultrasound system 10 (shown in FIG. 1).

A 3D processor sub-module 400 is also controlled by the user interface 30 and accesses the memory 390 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

The various embodiments and/or components, for example, the modules, or components and controllers therein, such as of the imaging system 10, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as an optical disk drive, solid state disk drive (e.g., flash RAM), and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” may include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

What is claimed is:

1. A method for controlling an ultrasound imaging system, said method comprising:
defining a sample volume gate on a two-dimensional (2D) ultrasound image, the sample volume gate defining a location at which flow is to be estimated;
automatically calculating a first signal-to-noise (SNR) with an initial aperture size and position;
automatically calculating a second SNR with a different aperture size and or position;
automatically comparing the first SNR with the first aperture to the second SNR with the second aperture;
automatically adjusting the steering angle and aperture size for the transmit and receive beams of an ultrasound probe based on the comparison.

2. The method of claim 1, further comprising:
generating a two-dimensional (2D) image of a blood vessel; and
defining the sample volume gate on the 2D image.

3. The method of claim 1, wherein automatically calculating the first SNR with the initial aperture size and position further comprises automatically calculating a ratio of one or more pixels identified as a signal to one or more pixels identified as noise for the initial aperture size and position.

4. The method of claim 1, wherein automatically calculating the second SNR with the different aperture size and position further comprises automatically calculating a ratio of one or more pixels identified as a signal to one or more pixels identified as noise for the second aperture size and position.

5. The method of claim 1, further comprising:
automatically calculating a SNR at a plurality of steering positions between the initial steering position and the second steering position;
automatically calculating a SNR at each of the plurality of steering positions;
automatically comparing the SNR acquired at each of the plurality of steering positions; and
automatically adjusting the steering angle and an aperture size of the ultrasound probe based on the comparison.

6. The method of claim 1, further comprising generating a spectrogram based on the sample volume gate.

7. The method of claim 1, further comprising:
receiving an input from an operator; and
automatically adjusting a position and an aperture size based on the input.

8. The method of claim 1, wherein the sample volume gate defines a portion of a blood vessel defining a blood flow to be estimated.

9. The method of claim 1, further comprising interleaving at least one ultrasound beam between the ultrasound beams emitted at the initial and second steering positions to reduce aliasing artifacts.

10. An ultrasound imaging system comprising:
an ultrasound probe comprising a transducer emitting ultrasound beams into a patient, the ultrasound probe acquiring a volume of ultrasound data comprising a blood vessel;
a user interface for defining s sample volume gate that encompasses at least a portion of the blood vessel; and
a processor configured to:
define a sample volume gate on a two-dimensional (2D) ultrasound image, the sample volume gate defining a location at which flow is to be estimated;
automatically calculate a signal-to-noise (SNR) with an initial aperture size and position;
automatically calculate a SNR with a different aperture size and or position;
automatically compare the SNR with the first aperture to the SNR with the second aperture;
automatically adjust the steering angle and aperture size for the transmit and receive beams of an ultrasound probe based on the comparison.

11. The ultrasound system of claim 10, wherein the processor is further configured to:
generate a two-dimensional (2D) image of the blood vessel; and
receive a user input that defines the sample volume gate on the 2D image.

12. The ultrasound system of claim 10, wherein to automatically calculate the first SNR with the initial aperture size and position, said processor is further programmed to automatically calculate a ratio of one or more pixels identified as a signal to one or more pixels identified as noise for the initial aperture size and position.

13. The ultrasound system of claim 10, wherein to automatically calculate the second SNR with the second aperture size and position, said processor is further programmed to automatically calculate a second ratio of one or more pixels identified as a signal to one or more pixels identified as noise for the second aperture size and position.

14. The ultrasound system of claim 10, wherein the processor is further configured to:
automatically calculate a SNR at a plurality of steering positions between the initial steering position and the second steering position;
automatically calculate a SNR at each of the plurality of steering positions;
automatically compare the SNR acquired at each of the plurality of steering positions; and
automatically adjust the steering angle and an aperture size of the ultrasound probe based on the comparison.

15. The ultrasound system of claim 10, wherein the processor is further configured to generate a spectrogram based on the sample volume gate.

16. The ultrasound system of claim 10, wherein the processor is further configured to:
receive an input from an operation; and
automatically adjust the steering angle and an aperture size of the ultrasound probe based on the input.

17. The ultrasound system of claim 10, wherein the processor is further configured to interleave at least one ultrasound beam between the ultrasound beams emitted at the initial and second steering positions to reduce aliasing artifacts.

18. A non-transitory computer readable medium programmed to instruct a processor to:
define a sample volume gate on a two-dimensional (2D) ultrasound image, the sample volume gate defining a location at which flow is to be estimated;
automatically calculate a signal-to-noise (SNR) with an initial aperture size and position;
automatically calculate a SNR with a different aperture size and or position;
automatically compare the SNR with the first aperture to the SNR with the second aperture;
automatically adjust the steering angle and aperture size for the transmit and receive beams of an ultrasound probe based on the comparison.

19. The non-transitory computer readable medium of claim 18 further programmed to:
generate a two-dimensional (2D) image of the blood vessel; and
receive a user input that defines the sample volume gate on the 2D image.

20. The non-transitory computer readable medium of claim 18 further programmed to:
automatically calculate a SNR at a plurality of steering positions between the initial steering position and the second steering position;
automatically calculate a SNR at each of the plurality of steering positions;
automatically compare the SNR's acquired at each of the plurality of steering positions; and
automatically adjust the steering angle and an aperture size of the ultrasound probe based on the comparison.
